(19) United States
(12) Patent Application Publication
(10) Pub. No.: US 2007/0074731 A1
(43) Pub. Date: Apr. 5, 2007

(54) Bio-implantable energy harvester systems and methods thereof

(75) Inventor: Michael D. Potter, Churchville, NY (US)

Correspondence Address: NIXON PEABODY LLP - PATENT GROUP, CLINTON SQUARE, P.O. BOX 31051, ROCHESTER, NY, 14603-1051, US

(73) Assignee: Nth Tech Corporation, Churchville, NY (US)

(21) Appl. No.: 11/243,662

(22) Filed: Oct. 5, 2005

(51) Int. Cl. A61N 1/378

(52) U.S. Cl. 128/899; 607/35; 320/137

(57) ABSTRACT

A bio-implantable power generation system includes at least one member with stored static electrical charge, at least two electrodes which are spaced from and on substantially opposing sides of the member, and a bio-attachment device connected to at least one of the electrodes for connecting the electrode to biological matter. The member is held in a fixed, spaced apart relationship with respect to one of the electrodes and the other one of the electrodes is movable with respect to the member and the one of the electrodes.

### FIELD OF THE INVENTION

This invention relates generally to power sources and, more particularly, to bio-implantable energy harvester systems and methods thereof.

### BACKGROUND OF THE INVENTION

There are a growing number of implanted medical devices which require miniaturized power sources. A variety of different types of power sources have been developed for these implantable devices. Although these power sources provide power for extended periods of time, they periodically still require replacement which involves further surgery on the subject.

### SUMMARY OF THE INVENTION

A bio-implantable power generation system in accordance with embodiments of the present invention includes at least one member with stored static electrical charge, at least two electrodes which are spaced from and on substantially opposing sides of the member, and a bio-attachment device connected to at least one of the electrodes for connecting the electrode to biological matter. The member is held in a fixed, spaced apart relationship with respect to one of the electrodes and the other one of the electrodes is movable with respect to the member and the one of the electrodes.

A method of making a bio-implantable power generation system in accordance with other embodiments of the present invention includes spacing at least two electrodes from and on substantially opposing sides of at least one member with stored static electrical charge. A bio-attachment device is connected to at least one of the electrodes for connecting the electrode to biological matter. The member is held in a fixed, spaced apart relationship with respect to one of the electrodes and the other one of the electrodes is movable with respect to the member and the one of the electrodes.

A method for generating power in accordance with other embodiments of the present invention includes moving one of at least two electrodes which are spaced from and on substantially opposing sides of at least one member with stored static electrical charge. The member is held in a fixed, spaced apart relationship with respect to one of the electrodes and the other one of the electrodes is movable with respect to the member and the one of the electrodes. At least one of the electrodes is connected to biological matter with a bio-attachment device. A potential is induced on the electrodes as a result of the moving and is output.

The present invention provides bio-implantable power systems which are compact, long lasting, reliable, and easily incorporated into biological subjects. This bio-implantable power systems provide a renewable source of power which will not require further surgery to replace. Instead, the present invention is able to effectively extract energy, and hence power, from the local biological environment in which it is implanted. By way of example only, this environment includes within the body of an animal or human.

### BRIEF DESCRIPTION OF DRAWINGS

FIG. 1 is a side, cross-sectional view of a portion of a bio-implantable energy harvester system in accordance with embodiments of the present invention;

FIG. 2 is a side, cross-sectional view of the bio-implantable energy harvester system shown in FIG. 1 implanted between a bone and tendon in a subject in a first position;

FIG. 3 is a side, cross-sectional view of the bio-implantable energy harvester system shown in FIG. 1 implanted between a bone and tendon in a subject in a second position;

FIG. 4 is a side, cross-sectional view of a bio-implantable energy harvester system in accordance with embodiments of the present invention implanted between a bone and tendon; and

FIG. 5 is a side, cross-sectional view of a portion of a bio-implantable energy harvester system in accordance with yet other embodiments of the present invention.

### DETAILED DESCRIPTION

A bio-implantable energy harvester system 10(1) in accordance with embodiments of the present invention is illustrated in FIGS. 1-3. The bio-implantable energy harvester system 10(1) includes a member 12(1) with a stored static electrical charge 14, electrodes 16 and 18, insulating layers 20 and 22, bio-attachment devices 24 and 26, an expandable housing 28 with a chamber 30, and a fluid 32 in the housing 28, although the system 10(1) can include other numbers and types of components and elements arranged in other configurations. The present invention provides a number of advantages including providing a compact, long lasting, and reliable bio-implantable power system which easily is incorporated into and utilizes natural movements of the biological subject to generate power.

Referring more specifically to FIGS. 1-3, the member 12(1) can hold a fixed, monopole charge 14 of electrons on the order of at least 1×10¹⁰ charges/cm², although the member 12(1) can store other types, amounts, and kinds of charge, such as a positive electrical charge. The member 12(1) includes dissimilar layers 34 and 36 of dielectric material which are seated against each other along an interface 38 where the fixed, monopole charge 14 is held, although the member 12(1) can comprise other numbers and types of layers in other configurations. For example, member 12(1) can comprise a single insulting layer which can hold the fixed, monopole charge 14 or multiple layers of dissimilar insulating layers which are seated against each other and can hold the fixed, monopole charge at one or more of the interfaces between these layers. The layer 34 is made of Si₃N₄ and layer 36 is made of SiO₂, although the layers 34 and 36 can be made of other types of dielectric materials, such as silicon oxide, silicon dioxide, silicon nitride, aluminum oxide, tantalum oxide, tantalum pentoxide, titanium oxide, titanium dioxide, barium strontium titanium oxide, zirconium oxide (ZrO₂) and niobium oxide (Nb₂O₅).

The electrodes 16 and 18 are substantially in alignment with each other and on opposite sides of member 12(1), although other numbers and types of conductors with other spacing, configuration, and alignments can be used. More specifically, the electrode 16 is spaced from and fixed with respect to member 12(1) and electrode 18 is spaced from and moveable with respect to member 12(1), although the member 12(1) and electrodes 16 and 18 can have other configurations and arrangements. The spacing is determined so that the electrodes 16 and 18 with respect to the member 12(1) have equal amounts of induced electrical charge at an initial state, although other spacing arrangements can be used. The position of the electrode 18 can be altered as a result of a movement to induce a difference in charge between the electrodes 16 and 18 which can be extracted as power, although other configurations can be used. The electrodes 16 and 18 can be coupled to a load (not shown), such as a pacemaker or other implanted medical device, to supply power extracted by the bio-implantable energy harvester system 10(1), although the electrodes 16 and 18 can be coupled to other types of systems and devices, such as a system or device which uses and/or stores the generated power.

The insulating layer 20 is secured to one surface of the electrode 16 and the insulating layer 22 is secured to one surface of the of electrode 18, although the surfaces of the electrodes 16 and 18 can be secured to other numbers and types of layers and the insulating layer 22 is optional and can be eliminated. Another surface of the insulating layer 20 is secured to one surface of the insulating layer 34 of the member 12(1) to hold the member 12(1) at a fixed distance from the electrode 16, although the member 12(1), electrode 16, and layer 20 can have other configurations and arrangements. Additionally, another surface of the insulating layer 22 faces, but is not secured to one surface of the insulating layer 36 of the member 12(1) to enable the another surface of the insulating layer 22 to rest against or be spaced from the one surface of the insulating layer 36, although the member 12(1), electrode 18, and layer 22 can have other configurations and arrangements and the insulating layer 36 is optional and can be eliminated. The insulating layer 20 is made of SiO₂ and the insulating layer 22 is a polymer, although the insulating layers 20 and 22 can be made of other types of materials. The insulating layer 22 is wider than the insulating layer 20 to control the amount of initial induced charge in electrode 18, although the insulating layers 20 and 22 can have other thicknesses and ratios with respect to each other.

The bio-attachment device 24 is used to secure the electrode 16 to a portion of a bone 40 and bio-attachment device 26 is used to secure the electrode 18 to a portion of a tendon 42, although the electrodes 16 and 18 can be secured in other manners with other types of systems and devices to other types of biological matter in the subject. The bio-attachment devices 24 and 26 are made of bio-scaffolding materials, although other types of materials can be used. During natural movements of the bone 40 with respect to the tendon 42 by the subject, the electrode 18 can be moved with respect to member 12(1) and electrode 16 to enable power to be extracted as explained in greater detail herein.

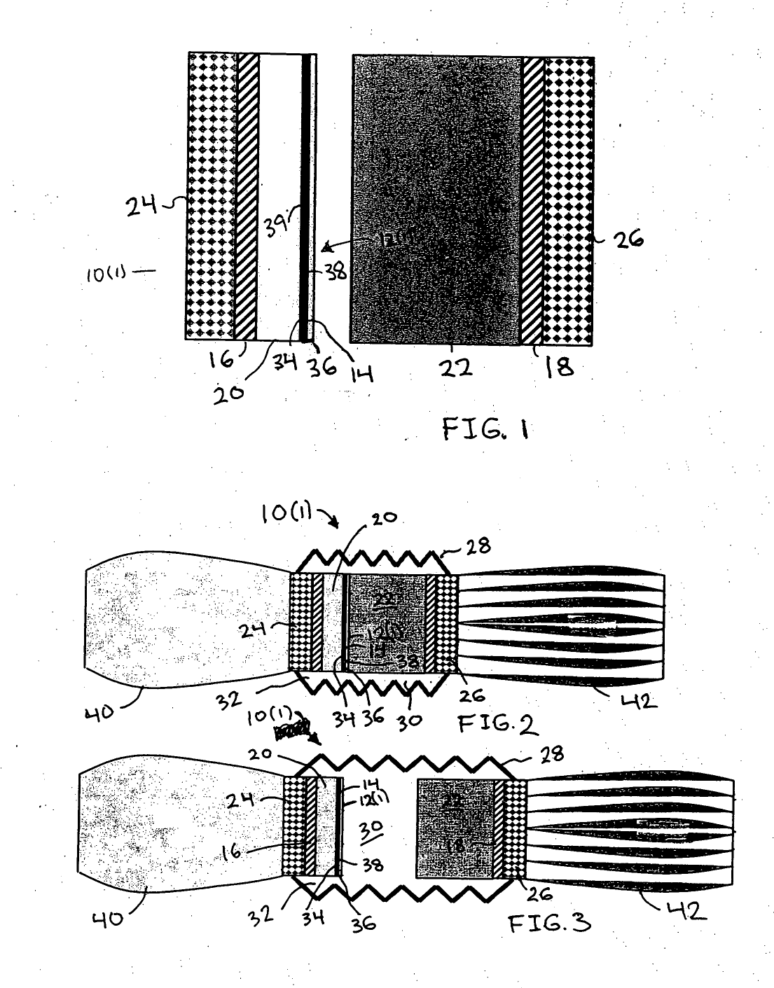

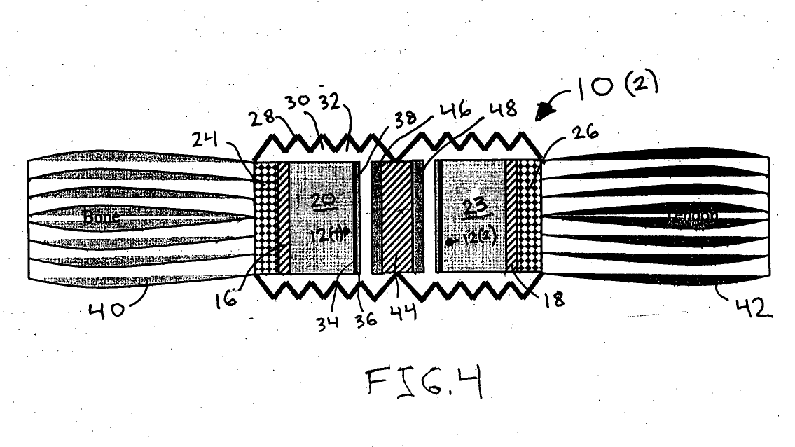

Referring to FIGS. 2-3, the expandable housing 28 has a bellows configuration which surrounds the member 12(1) and the electrodes 16 and 18 and is secured at opposing ends to the attachment devices 24 and 26 to form a sealed chamber 30, although the housing 28 could have other shapes and configurations and can be secured in other manners. The size of the housing 28 and of the chamber 30 can vary as required by the particular application. The chamber 30 can be filled with the fluid 32, such as de-ionized water, although other types of fluids and/or materials, including gases, can be used or the chamber 30 in housing 28 can be sealed in a vacuum. The fluid 32 has a relative dielectric constant of at least four, although the fluid 32 could have another dielectric constant and other properties. The fluid 32 in the chamber 30 increases the amount of power which can be generated by the bio-implantable energy harvester system 10(1) by at least three or four times compared to the amount of power which could be generated if the chamber 30 was filled with air.

Referring to FIG. 4, a bio-implantable energy harvester system 10(2) in accordance with other embodiments is shown. Elements in FIG. 4 which are like elements shown and described in FIGS. 1-3 will have like numbers and will not be shown and described in detail again here. In this embodiment, the insulating layer 23 is secured to one surface of the of electrode 18 and another surface of the insulating layer 23 is secured to another member 12(2), although the surfaces of the electrode 18 can be secured to other numbers and types of layers. The insulating layer 23 is made of silicon dioxide, although insulating layer 23 can be made of other types of materials. The member 12(2) comprises a pair of dissimilar insulating layers seated against each other with a fixed, monopole charge stored at the interface between the insulating layers. Like member 12(1) the member 12(2) can comprise other numbers and types of layers in other configurations.

An electrode 44 is connected to the housing 28 and is also located between and is spaced from the members 12(1) and 12(2), although the electrode 44 and members 12(1) and 12(2) could have other arrangements and configurations and the electrode 44 can be secured in other manners. An insulating layer 46 is on one surface of the electrode 44 and faces member 12(1) and another insulating layer 48 is on another surface of the electrode 44 and faces member 12(2), although insulating layers 46 and/or 48 are optional and may be eliminated. Electrode 16 and member 12(1) and electrode 18 and member 12(2) each can be brought toward and away from electrode 44 by natural movement of the subject's bone 40 and tendon 42 to induce a potential across electrodes 16 and 44 and across electrodes 18 and 44 which can be extracted to provide power, although again the bio-implantable energy harvester system 10(2) can be implanted between other biological matter in the subject. With this design additional power can be extracted from the bio-implantable energy harvester system 10(2).

Accordingly, by roughly doubling the size of the bio-implantable energy harvester system 10(2) by adding the additional member 12(2) with a fixed monopole charge and the electrode 44 configured in series as described in greater detail above, the bio-implantable energy harvester system 10(2) is able to extract about twice as much power from the same movement of the bone 40 and tendon 42 when compared to the bio-implantable energy harvester system 10(1). Additionally, the present invention can be scaled up to any multiple number of these combinations of these electrodes and members with a fixed monopole charge which are configured in series the same manner as described herein to proportionally increase the amount of power which can be generated.

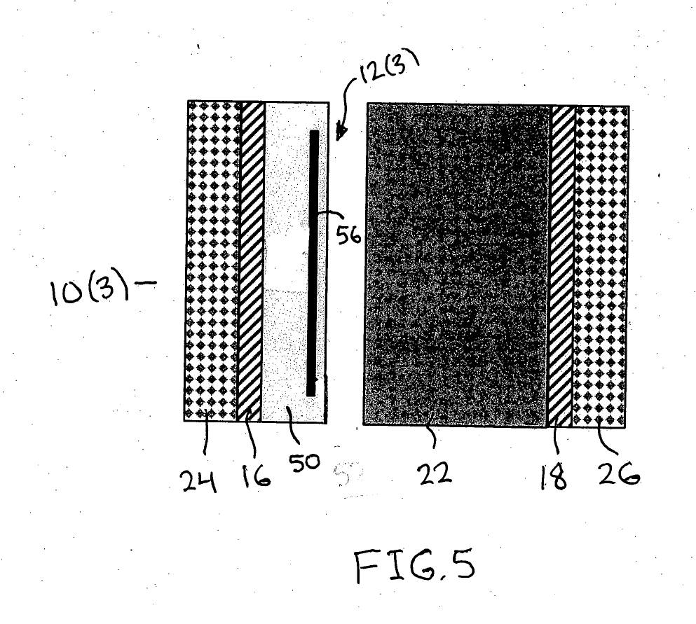

Referring to FIG. 5, a bio-implantable energy harvester system 10(3) in accordance with other embodiments is shown. Elements in FIG. 5 which are like elements shown and described in FIGS. 1-3 will have like numbers and will not be shown and described in detail again here. In this embodiment, member 12(3) includes a conducting layer 56, such as poly silicon, which is buried in an insulating layer 50, although the member 12(3) can comprise other numbers and types of layers in other arrangements and can be made of other materials. The member 12(3), which comprises the conducting layer 56, is a floating member which can hold a fixed, monopole charge 14 of electrons on the order of at least 1×10¹⁰ charges/cm², although the member 12(3) can store other types, amounts, and kinds of charge, such as a positive electrical charge.

A method for making the bio-implantable energy harvester system 10(1) in accordance with embodiments of the present invention is described below with reference to FIGS. 1-3. To make the bio-implantable energy harvester system 10(1), a fixed, monopole charge 14 of electrons on the order of at least 1×10¹⁰ charges/cm² is injected into the interface 38 between the dissimilar insulating layers 34 and 36 of member 12(1) which are secured together along interface 38, although other types of charge, such as a fixed monopole positive charge, could be stored and the fixed monopole charge can be injected to the interface in the member 12(1) in other manners. Additionally, the fixed monopole charge could be stored at other interfaces between the insulating layers, such as at interface 39 between insulating layer 20 and insulating layer 34.

The insulating layer 20 is secured to one surface of the electrode 16 and the insulating layer 22 is secured to one surface of the of electrode 18, although the surfaces of the electrodes 16 and 18 can be secured to other numbers and types of layers and again the insulating layer 22 is optional and may be eliminated. Another surface of the insulating layer 20 is secured to one surface of the insulating layer 34 of the member 12(1) to hold the member 12(1) at a fixed distance from the electrode 16, although the member 12(1), electrode 16, and layer 20 can have other configurations and arrangements and again the insulating layer 36 is optional and can be eliminated. Additionally, another surface of the insulating layer 22 faces, but is not secured to one surface of the insulating layer 36 of the member 12(1) to enable the another surface of the insulating layer 22 to rest against or be spaced from the one surface of the insulating layer 36, although the member 12(1), electrode 18, and layer 22 also can have other configurations and arrangements, such as eliminating insulating layers 22 and 36 and having electrode 18 be able to contact member 12(1).

The electrode 16 is secured to a portion of a bone 40 with bio-attachment device 24 and the electrode 18 to a portion of a tendon 42 with bio-attachment device 26, although the electrodes 16 and 18 can be secured in other manners to other types of biological material in the subject. During natural movements of the bone 40 with respect to the tendon 42 by the subject, the electrode 18 can be moved with respect to member 12(1) and electrode 16 to induce a potential which can be extracted as power.

The expandable housing 28 is secured around the member 12(1) and the electrodes 16 and 18 and to the attachment devices 24 and 26 to form a sealed chamber 30, although the housing 28 could be secured in other manners. The chamber 30 is filled with a fluid 32 which increases the amount of power which can be generated by the bio-implantable energy harvester system 10(1).

The method of making the bio-implantable energy harvester system 10(2) shown in FIG. 4 is the same as that for making the bio-implantable energy harvester system 10(1), except as described herein. The steps for making the bio-implantable energy harvester system 10(2) which are the same as those for making the bio-implantable energy harvester system 10(1), will not be described again here. To make the bio-implantable energy harvester system 10(2), a fixed, monopole charge 14 of electrons on the order of at least 1×10¹⁰ charges/cm² also is injected into the interface between the dissimilar layers of member 12(2), although the fixed monopole charge can be injected to the interface in the member 12(2) in other manners.

The surface of the insulating layer 23 which faces the electrode 44 is secured to a surface of the member 12(2) so that the member 12(2) is spaced from and held in a fixed relationship with respect to electrode 18, although other arrangements and configurations can be used. The electrode 44 is connected to the housing 28 and is between and spaced from the members 12(1) and 12(2), although the electrode 44 and members 12(1) and 12(2) could have other arrangements and configurations and the electrode 44 can be secured in other manners. An insulating layer 46 is connected to one surface of the electrode 44 which faces member 12(1) and another insulating layer 48 is connected to another surface of the electrode 44 which faces member 12(2), although other numbers and types of layers could be connected and each of the insulating layers 46 and 48 is optional and could be eliminated. Electrode 16 and member 12(1) and electrode 18 and member 12(2) can be brought toward and away from electrode 44 to induce a potential across electrodes 16 and 44 and across electrodes 18 and 44 which can be extracted to provide power, although the elements can be arranged to move in other manners.

The method of making the bio-implantable energy harvester system 10(3) shown in FIG. 5 is the same as that for making the bio-implantable energy harvester system 10(1), except as described herein. The steps for making the bio-implantable energy harvester system 10(3) which are the same as those for making the bio-implantable energy harvester system 10(1), will not be described again here.

To make the bio-implantable energy harvester system 10(3), the insulating layer 50 is formed around the conducting layer 56. A fixed, monopole charge 14 of electrons on the order of at least 1×10¹⁰ charges/cm² is injected into the conducting layer 56 which comprises the floating member 12(3), although other types of charge, such as a fixed monopole positive charge, could be stored and the fixed monopole charge can be injected to the conducting layer 56 in the member 12(3) in other manners. The insulating layer 50 is secured to one surface of the electrode 16, although the surfaces of the electrode 16 can be secured to other numbers and types of layers.

The operation of the bio-implantable energy harvester system 10(1) in accordance with embodiments will be described with reference to FIGS. 1-3. With natural movements of the subject with the bio-implantable energy harvester system 10(1), the bone 40 moves with respect to the tendon 42. This movement of the bone 40 and tendon 42 causes the electrode 18 to move with respect to the member 12(1) which has the fixed monopole charge and the electrode 16 and induces a potential across the electrodes 16 and 18. This induced potential can be output to other implanted medical devices in the subject to provide power and/or could be stored for future use in a device in the subject. If a fluid 32 is introduced in the chamber 30 of the housing 28, then greater levels of charge can be induced in electrode 18 which increases the output power.

The operation of the bio-implantable energy harvester system 10(2) with reference to FIG. 4 is the same as that for the bio-implantable energy harvester system 10(1), except as described herein. Again, with natural movements of the subject with the bio-implantable energy harvester system 10(2), the bone 40 moves with respect to the tendon 42. This movement of the bone 40 and tendon 42 causes the electrode 16 with the member 12(1) and the electrode 18 with the member 12(2) to move with respect to the electrode 44 and induces a potential across the electrodes 16 and 44 and also across the electrodes 18 and 44. This induced potential can be output to other implanted medical devices in the subject to provide power and/or could be stored for future use in a device in the subject. Accordingly, as illustrated by this embodiment and discussed earlier by proportionally increasing the number of electrodes and members with fixed monopole charge arranged in series in the configurations described herein, the amount of power which can be extracted is increased. Again, if a fluid 32 is introduced in the chamber 30 of the housing 28, then greater levels of charge can be induced in electrodes 16 and 44 and in electrodes 18 and 44.

The operation of the bio-implantable energy harvester system 10(3) is the same as that for the bio-implantable energy harvester system 10(1) except that a floating member 12(3) is used to hold the fixed, monopole charge and thus will not be described again here.

Accordingly, the present invention is directed to bio-implantable power systems which are compact, long lasting, reliable, and easily incorporated into biological subjects. The present invention is able to effectively extract energy, and hence power, from the local biological environment in which it is implanted and therefore will not require replacement during the life of the biological subject in which the power system is implanted.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefor, is not intended to limit the claimed processes to any order except as may be specified in the claims. Accordingly, the invention is limited only by the following claims and equivalents thereto.

What is claimed is:

1. A bio-implantable power generation system comprising:
at least one member with stored static electrical charge;
at least two electrodes which are spaced from and on substantially opposing sides of the member; and
a bio-attachment device connected to at least one of the electrodes for connecting the electrode to biological matter, wherein the member is held in a fixed, spaced apart relationship with respect to one of the electrodes, the other one of the electrodes is movable with respect to the member and the one of the electrodes.

2. The system as set forth in claim 1 further comprising an expandable housing which surrounds at least a portion of the member and the electrodes.

3. The system as set forth in claim 2 further comprising one or more fluids in the expandable housing.

4. The system as set forth in claim 3 wherein the one or more fluids has a dielectric constant of at least four.

5. The system as set forth in claim 1 further comprising at least one insulating layer which is between the member and the one of the electrodes which is held in a fixed, spaced apart relationship with respect to the member.

6. The system as set forth in claim 5 further comprising at least one other insulating layer which is on the other one of the electrodes which is movable with respect to the member.

7. The system as set forth in claim 1 wherein the stored static electrical charge in the member is a monopole charge.

8. The system as set forth in claim 1 wherein the stored static electrical charge is on the order of at least 1×10¹⁰ charges/cm².

9. The system as set forth in claim 1 wherein the member comprises two or more dielectric layers and the stored static electrical charge is substantially stored at an interface between the dielectric layers.

10. The system as set forth in claim 9 wherein at least two of the two or more dielectric layers are made of dissimilar materials.

11. The system as set forth in claim 1 wherein the member comprises a floating member with the stored static electric charge.

12. The system as set forth in claim 1 further comprising:
at least one other member with stored static electrical charge;
at least two other electrodes which are spaced from and on substantially opposing sides of the other member; and
a bio-attachment device connected to at least one of the other electrodes for connecting the other one of the electrodes to biological matter, wherein the other member is held in a fixed, spaced apart relationship with respect to one of the other electrodes, the other one of the other electrodes is movable with respect to the member and the other one of the other electrodes.

13. The system as set forth in claim 12 further comprising at least one other insulating layer which is between the other member and the one of the other electrodes which is held in a fixed, spaced apart relationship with respect to the other member.

14. The system as set forth in claim 13 further comprising at least one other insulating layer which is on the other one of the electrodes which is movable with respect to the member.

15. The system as set forth in claim 12 wherein one of the electrodes and one of the other electrodes comprises the same electrode.

16. The system as set forth in claim 12 wherein the stored static electrical charge in the other member is a monopole charge.

17. The system as set forth in claim 12 wherein the stored static electrical charge is on the order of at least 1×10¹⁰ charges/cm².

18. The system as set forth in claim 12 wherein the other member comprises two or more dielectric layers and the stored static electrical charge is substantially stored at an interface between the dielectric layers.

19. The system as set forth in claim 18 wherein at least two of the two or more dielectric layers are made of dissimilar materials.

20. The system as set forth in claim 12 wherein the other member comprises a floating member with the stored static electric charge.

21. A method of making a bio-implantable power generation system, the method comprising:
spacing at least two electrodes from and on substantially opposing sides of at least one member with stored static electrical charge; and
connecting a bio-attachment device to at least one of the electrodes for connecting the electrode to biological matter, wherein the member is held in a fixed, spaced apart relationship with respect to one of the electrodes, the other one of the electrodes is movable with respect to the member and the one of the electrodes.

22. The method as set forth in claim 21 further comprising surrounding at least a portion of the member and the electrodes with an expandable housing.

23. The method as set forth in claim 22 further comprising placing one or more fluids in the expandable housing.

24. The method as set forth in claim 23 wherein the one or more fluids has a dielectric constant of at least four.

25. The method as set forth in claim 21 further comprising providing at least one insulating layer which is between the member and the one of the electrodes which is held in a fixed, spaced apart relationship with respect to the member.

26. The method as set forth in claim 25 further comprising providing at least one other insulating layer which is on the other one of the electrodes which is movable with respect to the member.

27. The method as set forth in claim 21 wherein the stored static electrical charge in the member is a monopole charge.

28. The method as set forth in claim 21 wherein the stored static electrical charge is on the order of at least 1×10¹⁰ charges/cm².

29. The method as set forth in claim 21 wherein the member comprises two or more dielectric layers and the stored static electrical charge is substantially stored at an interface between the dielectric layers.

30. The method as set forth in claim 29 wherein at least two of the two or more dielectric layers are made of dissimilar materials.

31. The method as set forth in claim 21 wherein the member comprises a floating member with the stored static electric charge.

32. The method as set forth in claim 21 further comprising:
at least one other member with stored static electrical charge;
at least two other electrodes which are spaced from and on substantially opposing sides of the other member from each other and are at least partially in alignment with each other; and
a bio-attachment device connected to at least one of the other electrodes for connecting the other electrodes to biological matter, wherein the other member is held in a fixed, spaced apart relationship with respect to one of the other electrodes, the other one of the other electrodes is movable with respect to the member and the other one of the other electrodes.

33. The method as set forth in claim 32 further comprising at least one other insulating layer which is between the other member and the one of the other electrodes which is held in a fixed, spaced apart relationship with respect to the other member.

34. The method as set forth in claim 32 further comprising at least one other insulating layer which is on the other one of the electrodes which is movable with respect to the member.

35. The method as set forth in claim 32 wherein one of the electrodes and one of the other electrodes comprises the same electrode.

36. The method as set forth in claim 32 wherein the stored static electrical charge in the other member is a monopole charge.

37. The method as set forth in claim 32 wherein the stored static electrical charge is on the order of at least 1×10¹⁰ charges/cm².

38. The method as set forth in claim 32 wherein the other member comprises two or more dielectric layers and the stored static electrical charge is substantially stored at an interface between the dielectric layers.

39. The method as set forth in claim 38 wherein at least two of the two or more dielectric layers are made of dissimilar materials.

40. The method as set forth in claim 32 wherein the other member comprises a floating member with the stored static electric charge.

41. A method for generating power, the method comprising:
moving one of at least two electrodes which are spaced from and on substantially opposing sides of at least one member with stored static electrical charge, wherein the member is held in a fixed, spaced apart relationship with respect to one of the electrodes, the other one of the electrodes is movable with respect to the member and the one of the electrodes and wherein at least one of the electrodes is connected to biological matter with a bio-attachment device;
inducing a potential on the electrodes as a result of the moving; and
outputting the induced potential.

42. The method as set forth in claim 41 wherein at least a portion of the member and the electrodes are surrounded by an expandable housing.

43. The method as set forth in claim 42 wherein one or more fluids are in the expandable housing.

44. The method as set forth in claim 43 wherein the one or more fluids has a dielectric constant of at least four.

45. The method as set forth in claim 41 wherein at least one insulating layer is between the member and the one of the electrodes which is held in a fixed, spaced apart relationship with respect to the member.

46. The method as set forth in claim 45 wherein at least one other insulating layer is on the other one of the electrodes which is movable with respect to the member.

47. The method as set forth in claim 45 wherein the stored static electrical charge in the member is a monopole charge.

48. The method as set forth in claim 41 wherein the stored static electrical charge is on the order of at least 1×10¹⁰ charges/cm².

49. The method as set forth in claim 41 wherein the member comprises two or more dissimilar dielectric layers and the stored static electrical charge is substantially stored at an interface between the dielectric layers.

50. The method as set forth in claim 49 wherein at least two of the two or more dielectric layers are made of dissimilar materials.

51. The method as set forth in claim 41 wherein the member comprises a floating member with the stored static electric charge.

52. The method as set forth in claim 41 further comprising:
moving one of at least two other electrodes which are spaced from and on substantially opposing sides of at least one other member with stored static electrical charge, wherein the other member is held in a fixed, spaced apart relationship with respect to one of the other electrodes, the other one of the other electrodes is movable with respect to the other member and the one of the other electrodes and wherein at least one of the other electrodes is connected to other biological matter with another bio-attachment device;
inducing additional potential on the other electrodes as a result of the moving; and
outputting the additional induced potential.

53. The method as set forth in claim 52 wherein at least one other insulating layer is between the other member and the one of the other electrodes which is held in a fixed, spaced apart relationship with respect to the other member.

54. The method as set forth in claim 53 wherein at least one other insulating layer is on the other one of the electrodes which is movable with respect to the member.

55. The method as set forth in claim 54 wherein one of the electrodes and one of the other electrodes comprises the same electrode.

56. The method as set forth in claim 52 wherein the stored static electrical charge in the other member is a monopole charge.

57. The method as set forth in claim 52 wherein the stored static electrical charge is on the order of at least 1×10¹⁰ charges/cm².

58. The method as set forth in claim 52 wherein the other member comprises two or more dielectric layers and the stored static electrical charge is substantially stored at an interface between the dielectric layers.

59. The method as set forth in claim 58 wherein at least two of the two or more dielectric layers are made of dissimilar materials.

60. The method as set forth in claim 52 wherein the other member comprises a floating member with the stored static electric charge.
